(19) United States
(12) Patent Application Publication
(10) Pub. No.: US 2026/0207068 A1
(43) Pub. Date: Jul. 23, 2026

(54) METHOD FOR MONITORING BLOOD PRESSURE

(71) Applicant: GE Precision Healthcare LLC, Waukesha, WI (US)

(72) Inventor: Samuli Ville Valtteri Karvinen, Helsinki (FI)

(21) Appl. No.: 19/035,477

(22) Filed: Jan. 23, 2025

(51) Int. Cl. A61B 5/024; A61B 5/021

(52) U.S. Cl. CPC A61B 5/02422; A61B 5/02116; A61B 5/02125; A61B 2560/0228

(57) ABSTRACT

Methods and systems are provided herein for measuring a blood pressure (BP) of a subject when continuous monitoring is desired, and a traditional cuff-based blood pressure measurement system may be restrictive. The proposed systems and methods measure the BP using dual photoplethysmography (PPG) sensors integrated into the cuff, which can dramatically reduce the frequency of cuff inflation while providing continuous BP monitoring. The dual PPG sensors monitor a BP of a patient by calculating a Pulse Wave Velocity (PWV) of blood in a blood vessel of the patient based on a Pulse Transit Time (PTT), and an PPG Intensity Ratio (PIR). The patient's BP may be measured continuously based on these physiological parameters, with the cuff serving as a reference to periodically recalibrate the system automatically, thereby enhancing accuracy and reducing a demand for frequent cuff inflation.

### FIELD

Embodiments of the subject matter disclosed herein relate to blood pressure monitoring.

### BACKGROUND

Blood pressure (BP) is a measured vital sign reflecting the pressure in a person's arteries during a cardiac cycle, for ensuring adequate blood flow, and therefore oxygen and nutrient delivery, to all body tissues and organs. Appropriate BP levels are necessary for maintaining organ perfusion and function, and deviations from a normal range can indicate or lead to serious health issues such as organ damage, stroke, and heart disease. As a result, BP may be continuously monitored and managed in real-time in critical care settings and for patients with conditions affecting cardiovascular health. Typically, blood pressure is measured using a cuff-based instrument, such as a digital blood pressure monitor for home use, a manually operated sphygmomanometer used by healthcare professionals, or an ambulatory blood pressure monitor for patient monitoring at regular intervals. However, continuously monitoring the BP may entail repeatedly inflating the cuff of the cuff-based instrument, which may be uncomfortable for a patient.

### BRIEF DESCRIPTION

In one embodiment, a system for monitoring blood pressure (BP) of a patient comprises a BP measurement device including a cuff; at least two photoplethysmography (PPG) sensors arranged in the cuff such that when the cuff is fastened around an arm of the patient, the at least two PPG sensors are positioned to sense a flow of blood in a brachial artery of the patient; and a controller and a memory storing instructions that when executed, cause the controller to receive PPG signals from the at least two PPG sensors; calculate an instantaneous pulse pressure (BP) of the patient based on the received PPG signals; display an arterial BP waveform generated from a plurality of instantaneous BP measurements on a display device; and recalibrate the PPG signals based on a reference signal measured by the BP measurement device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a block diagram illustrating one embodiment of a multi-wavelength pulse oximetry system;

FIG. 2 is an image showing an exemplary placement of PPG sensors in a cuff on an arm of a human subject;

FIG. 3 is a flowchart showing an exemplary high-level method for calculating and displaying a continuous waveform of blood pressure from signals from PPG sensors;

FIG. 4 is a flowchart showing an exemplary method for generating a calibration function for calibrating the PPG signals;

FIG. 5 is a flowchart showing an exemplary method for calibrating the PPG signals during monitoring of a BP of a patient;

FIG. 6 is a flowchart showing an exemplary method for calculating an instantaneous blood pressure of the patient from PPG signals;

FIG. 7 is an exemplary graph showing a plot of a calibration curve for calibrating the PPG signals;

FIG. 8 is an exemplary graph showing a plot of a pulse pressure of a patient over a cardiac cycle;

FIG. 9 is an exemplary graph of a derived pulse pressure estimation model; and

FIG. 10 shows a graphical comparison to two approaches to performing a polynomial regression of cardiac waveform data.

### DETAILED DESCRIPTION

Blood pressure (BP) can typically be measured using a sphygmomanometer, a digital blood pressure monitor, or an ambulatory blood pressure monitor. An inflatable cuff is placed on an arm of the subject, which is inflated to a target pressure. Blood pressure measurements are then taken as the cuff is deflated. However, a disadvantage of cuff-based BP measurement methods, including the use of a sphygmomanometer, a digital blood pressure monitor, and an ambulatory blood pressure monitor, is that they may be cumbersome and may rely on a trained professional to obtain accurate readings. Additionally, such traditional methods may not be suitable for continuous monitoring, for example, when managing patients in clinical wards, especially patients with chronic conditions like hypertension or those at risk of cardiovascular events.

To address this issue, systems and methods are provided herein for measuring a BP of a subject when continuous monitoring is desired, and a traditional cuff-based blood pressure measurement system may be restrictive. Rather than relying on the sphygmomanometer for the BP measurements, the proposed systems and methods measure the BP using photoplethysmography (PPG) sensors.

PPG relates to the use of optical signals transmitted through or reflected by blood-perfused tissues for monitoring a physiological parameter of a subject (also referred to as a patient herein). In this technique, one or more emitters are used to direct light at a tissue of the subject, and one or more detectors are used to detect the light that is transmitted through or reflected by the tissue. The volume of blood of the tissue affects the amount of light that is transmitted or reflected, which is output as a PPG signal. As the blood volume in a tissue changes with each heartbeat, the PPG signal also varies with each heartbeat.

A PPG device comprises a computerized measuring unit and a probe attached to a patient. The probe includes a light source for sending an optical signal through tissue of the patient and a photo detector for receiving the signal transmitted through or reflected from the tissue. On the basis of the transmitted and received signals, light absorption by the tissue may be determined. During each cardiac cycle, light absorption by the tissue varies cyclically. During the diastolic phase, absorption is caused by venous blood, non-pulsating arterial blood, cells and fluids in tissue, bone, and pigments. The level of light transmitted at end of the diastolic phase is typically referred to as the “DC component” of the total light transmission. During the systolic phase, there is an increase in light absorption (e.g., a decrease in transmitted light) compared with the diastolic phase due to the inflow of arterial blood into the tissue on which the probe is attached. The level of light transmitted during the systolic phase is typically referred to as the “AC component” of the total light transmission. The amount of light transmitted through a tissue varies according to changes in blood volume at the site. Specifically, the amount of light transmitted through vascular bed decreases during systole (e.g., more light is absorbed) and increases during diastole, resulting in periodic PPG waveforms (e.g., the AC/DC component). Multiple physiological parameters can be extracted from the PPG waveforms.

The methods disclosed herein rely on dual PPG sensors that may be integrated into a cuff of a traditional cuff-based measurement system, which can dramatically reduce the frequency of cuff inflation while providing continuous BP monitoring. As described in greater detail below, the dual PPG sensors are used to monitor a BP of a patient by calculating three physiological parameters: a Pulse Wave Velocity (PWV) of blood in the patient's blood vessel, a Pulse Transit Time (PTT) used to calculate the PWV, and a PPG Intensity Ratio (PIR), which may provide a more accurate and reliable blood pressure reading compared to relying solely on PTT or PWV. The patient's BP may be measured continuously based on these physiological parameters, with the cuff serving to provide a reference signal for recalibrating the system automatically under certain circumstances, for example, when a diastolic PPG increases above a threshold value, thereby enhancing accuracy and reducing a demand for frequent cuff inflation.

By reducing the demand for frequent cuff inflation, the monitoring process may be more comfortable and less intrusive for patients during continuous monitoring in a ward setting. Blood pressure may be tracked continuously in real time, providing valuable data for ongoing health assessment and early intervention in clinical environments. Additionally, by combining data from the two PPG sensors and integrating the physiological parameters PWV and PIR into the same theoretical model, the system may increase an accuracy and reliability of blood pressure estimation, with automatic recalibration using the cuff. As a result, blood pressure monitoring is simplified, thereby improving workflow efficiency in the ward.

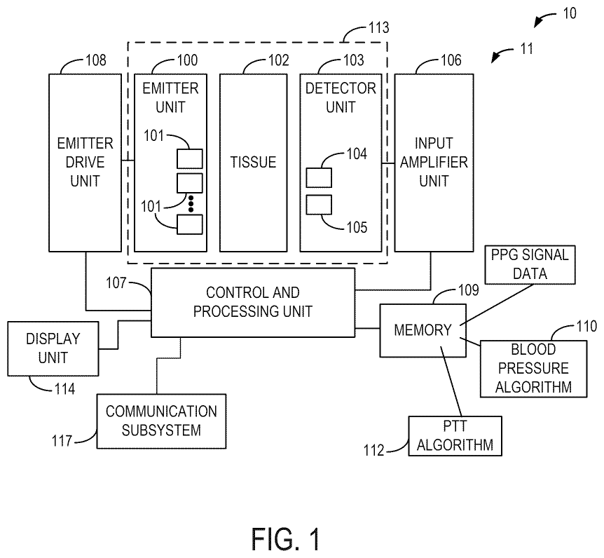

FIG. 1 is a block diagram of one embodiment of a multi-parametric PPG system 10, which may be capable of measuring various parameters, including Blood Pressure, Heart Rate, SpO2, Arterial Stiffness, etc. Light transmitted from an emitter unit 100 passes into patient tissue 102. The emitter unit includes multiple light sources 101, such as light-emitting diodes (LEDs), with each light source having a dedicated wavelength. Each wavelength forms one measurement channel on which PPG waveform data are acquired. The number of sources/wavelengths is at least two. When assessing a blood pressure waveform, to ensure adequate penetration depth and minimize the effect of blood oxygen levels, infrared light may be used.

The light transmitted through the tissue 102 is received by a detector unit 103, which comprises two photo detectors 104 and 105 in this example. For example, photo detector 104 may be a silicon photodiode, and photo detector 105 may be a second silicon photodiode with different spectral characteristics or an indium gallium arsenide (InGaAs) photodiode. The emitter and detector units form a probe detector subunit 113 of the PPG system 10. The photo detectors convert the optical signals received into electrical pulse trains and feed them to an input amplifier unit 106. The amplified measurement channel signals are further supplied to a control and processing unit 107, which executes instructions stored in memory to convert the signals into digitized format for each wavelength channel.

The control and processing unit 107 further controls an emitter drive unit 108 to alternately activate the light sources. As mentioned above, each light source is typically illuminated several hundred times per second. With each light source being illuminated at such a high rate compared to the pulse rate of the patient, the control and processing unit 107 obtains a high number of samples at each wavelength for each cardiac cycle of the patient. The value of these samples varies according to the cardiac cycle of the patient, the variation being caused by the arterial blood.

The input amplifier unit 106, the control and processing unit 107, the emitter drive unit 108, and probe detector subunit 113 collectively form a probe 11. As used herein, the term “probe” may refer to the probe 11 and the attachment parts that attach the optical components, the probe 11, to the tissue site. The term “PPG sensor” may refer to a unit comprising a probe, an analog front end, and a signal processing unit that calculates various blood characteristics. In a multi-parameter body area network system, the system typically represents a set of multiple sensors, e.g., the different physiological parameter measurements. Therefore, the whole measurement system may comprise of several sensors and their associated probes, and the sensors may communicate to a common hub in which the parameters' information is integrated.

The digitized PPG signal data at each wavelength may be stored in a memory 109 of the control and processing unit 107 before being processed further according to non-transitory instructions (e.g., algorithms) executable by the control and processing unit 107 to obtain physiological parameters. For example, memory 109 may comprise a suitable data storage medium, for example, a permanent storage medium, removable storage medium, and the like. Additionally, memory 109 may be a non-transitory storage medium. In some examples, the system 10 may include a communication subsystem 117 operatively coupled to one or more remote computing devices, such as hospital workstations, smartphones, and the like. The communication subsystem 117 may enable the output from the detector units (e.g., the digitized PPG signal data) to be sent to the one or more remote computing devices for further processing and/or the communication subsystem may enable the output from the algorithms discussed below (e.g., determined physiological parameters) to be sent to the remote computing devices. The communication subsystem 117 may include wired and/or wireless communication devices compatible with one or more different communication protocols. As non-limiting examples, the communication subsystem 117 may be configured for communication via a wireless telephone network, a local- or wide-area network, and/or the Internet.

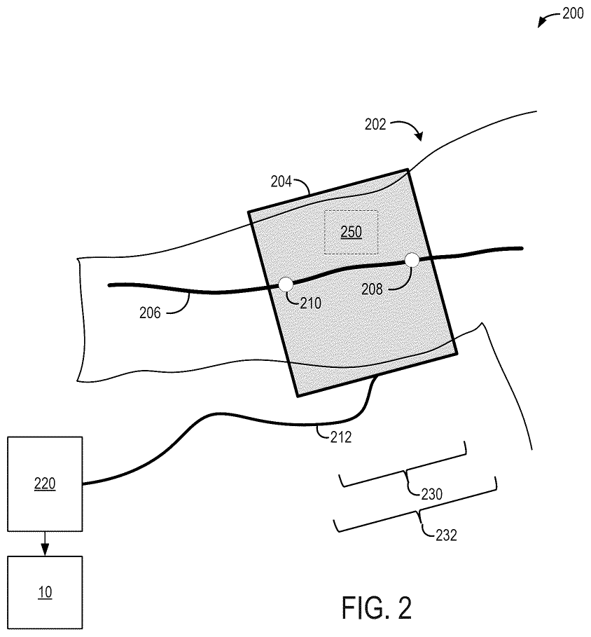

Algorithms may utilize the same digitized signal data and/or results derived from the algorithms and stored in the memory 109, for example. For example, for a determination of pulse transit time (PTT), the control and processing unit 107 is adapted to execute one or more PTT algorithms 112, which may also be stored in the memory 109 of the control and processing unit 107. A blood pressure algorithm 110 may also be stored in the memory 109 for determining BP. The use of such algorithms will be described in more detail below with respect to FIGS. 2-10. The obtained physiological parameters and waveforms may be shown on a screen of a display unit 114. Further, in some examples, the control and processing unit, memory, and/or other subsystems may be located remotely from the rest of the sensor on a separate device, and the signal data from the detector units may be sent to the separate device for processing.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

Referring now to FIG. 2, a sensor placement diagram 200 shows a placement of two PPG sensors of PPG system 10 described above in reference to FIG. 1, on a patient. Specifically, a first PPG sensor 208 and a second PPG sensor 210 may be arranged in or on a cuff 204 of a sphygmomanometer 220. Cuff 204 may be fastened around an arm 202 of the patient, such that first PPG sensor 208 and second PPG sensor 210 are positioned over a brachial artery 206 of the patient (e.g., such that first PPG sensor 208 and second PPG sensor 210 are positioned to sense a flow of blood in a brachial artery of the patient). First PPG sensor 208 may be referred to herein as a proximal PPG sensor (e.g., proximal to a heart of the patient, with respect to second PPG sensor 210), and second PPG sensor 210 may be referred to herein as a distal PPG sensor (e.g., distal to the heart, with respect to first PPG sensor 210). A distance 230 between first PPG sensor 208 and second PPG sensor 210 may be determined based on a maximum sampling rate of first PPG sensor 208 and second PPG sensor 210 and dimensions of arm 202. In one example, distance 230 is 10 cm. In general, distance 230 may be a maximum distance permitted by a length 232 of cuff 204, since the sampling rate is inversely proportional to distance 230. That is, as distance 230 increases, the sampling rate relied on to calculate the time difference (PTT) between the detection of the systolic peak of a cardiac cycle at first (proximal) PPG sensor 208 and the detection of the systolic peak at second (distal) PPG sensor 210 decreases. An optimal distance may be determined by measuring the distance between two most prominent PPG locations along brachial artery 206. The two most prominent PPG locations are likely to be the closest to the skin surface for high quality signal acquisition. In one example, the first and second PPG sensors 208 and 210, respectively, are positioned at the medial side of mid-upper arm and medial to the biceps tendon above the cubital fossa, respectively, with the distance being between 5-15 cm. In other embodiments, additional PPG sensors may be used.

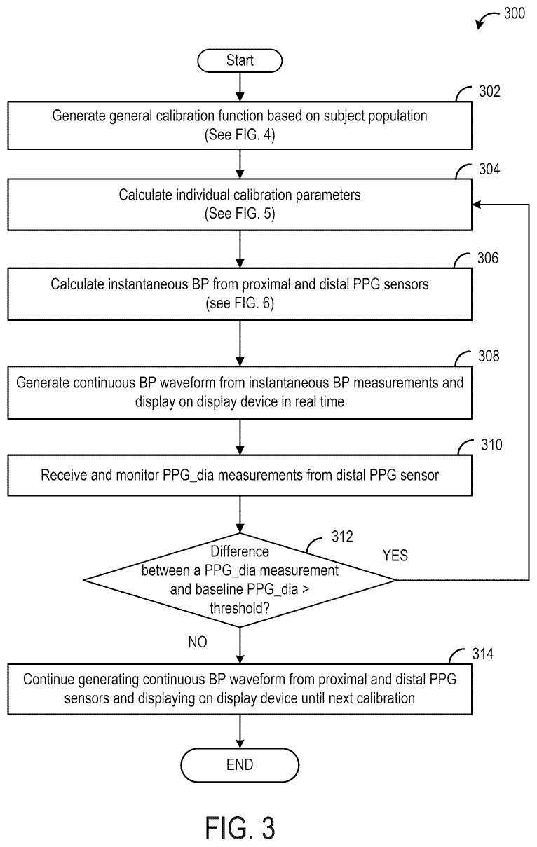

Conventionally, a BP of the patient may be measured using sphygmomanometer 220. To measure the BP, cuff 204 may be inflated until a pulse can no longer be sensed by sphygmomanometer 220. Cuff 204 may then be deflated until the pulse is sensed by sphygmomanometer 220. However, sphygmomanometer 220 may not be suitable for constant monitoring of the BP, due to a discomfort of repeatedly inflating and deflating cuff 204. However, the BP may also be estimated based on PPG signals generated by first PPG sensor 208 and second PPG sensor 210, in accordance with the method described in reference to FIG. 3. The PPG signals generated by first PPG sensor 208 and second PPG sensor 210 may be transmitted by a wire in a cord 212 connecting cuff 204 to sphygmomanometer 220, which may be communicatively coupled to PPG system 10. The PPG signals may be received and processed at PPG system 10, and a BP waveform based on the PPG signals may be displayed on a display device of PPG system 10 (e.g., display unit 114). The BP of the patient may be monitored via the display device. The estimation of the BP based on the PPG signals may not rely on repeatedly inflating and deflating cuff 204, which may increase a comfort of the patient. However, as described in greater detail below, the PPG signals may be periodically calibrated based on a BP measurement made using sphygmomanometer 220.

In various embodiments, power for first PPG sensor 208 and second PPG sensor 210 may be supplied by a battery 250, which may be positioned at a lateral side of cuff 204, meaning, on an opposite side of cuff 204 as first PPG sensor 208 and second PPG sensor 210. Such a placement may avoid obstructing the patient, enhance wireless signal quality, and prevent the patient's side from heating up. In other embodiments, battery 250 may be provided at a different location.

Turning now to FIG. 3, a high-level flowchart is shown illustrating a method 300 for calculating and displaying a continuous waveform of a BP of a patient from PPG signals measured by PPG sensors of a PPG system, such as PPG system 10 shown in FIGS. 1 and 2. More specifically, steps of method 300 and the other methods described herein may be executed by a control and processing unit of the PPG system (such as control and processing unit 107) according to instructions stored on a non-transitory memory of the PPG system (e.g., memory 109 shown in FIG. 1) in combination with the various signals received at the control and processing unit from the system components and actuation signals sent from the system controller to the display device (e.g., display unit 114).

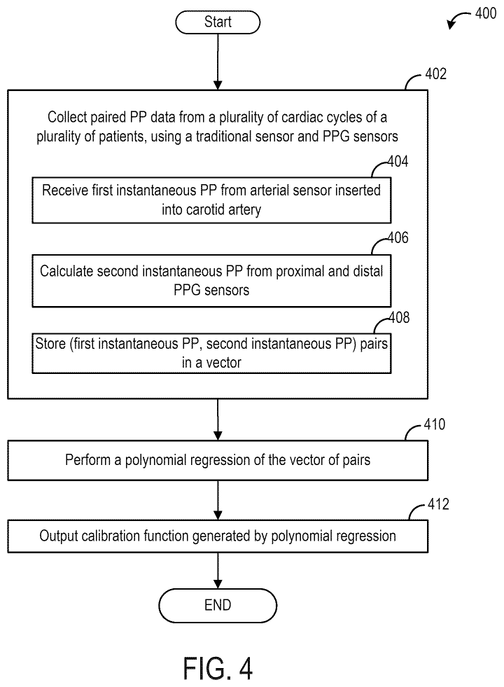

Method 300 begins at 302, where method 300 includes generating a general calibration function for calibrating a theoretical pulse pressure model so that a predicted theoretical brachial pulse pressure (derived from the PIR and the PWV) is adjusted to match observed values (e.g., a true brachial pulse pressure measured using an invasive sensor inside the brachial artery). The PPG sensors may be integrated into a cuff of a sphygmomanometer, as described in reference to FIG. 2. The PPG sensors may include a proximal PPG sensor positioned at a first side of the cuff closest to a body of the patient (e.g., proximal PPG sensor 208), and a distal PPG sensor positioned at a second side of the cuff farthest from the body (e.g., distal PPG sensor 210). Either or both of the proximal PPG sensor and the distal PPG sensor may be calibrated during monitoring of the BP of the patient using the general calibration function. Generating the general calibration function is described in greater detail below in reference to FIG. 4.

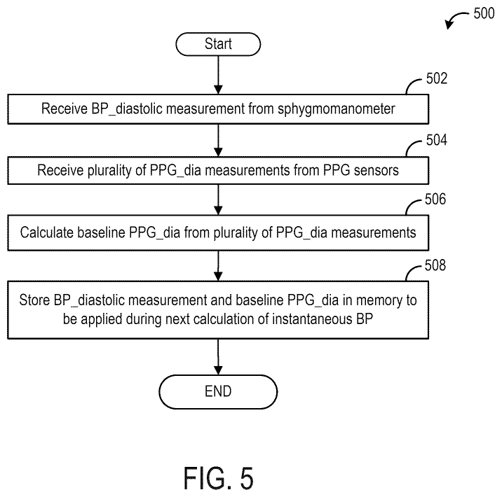

At 304, method 300 includes calculating individual calibration parameters that will be used for the calculation of an instantaneous BP at step 306. Calculating the individual calibration parameters includes measuring a BP of the patient, using the sphygmomanometer. The individual calibration parameters may be calculated prior to beginning monitoring of the BP of the patient, and the individual calibration parameters may be periodically recalculated during monitoring the BP for calibration during the instantaneous BP calculation. During the calibration, baseline diastolic PPG (PPG_{dia}) amplitudes measured by the proximal PPG sensor and the distal PPG sensor may be measured. The measurement of the individual calibration parameters is described in greater detail below in reference to FIG. 5.

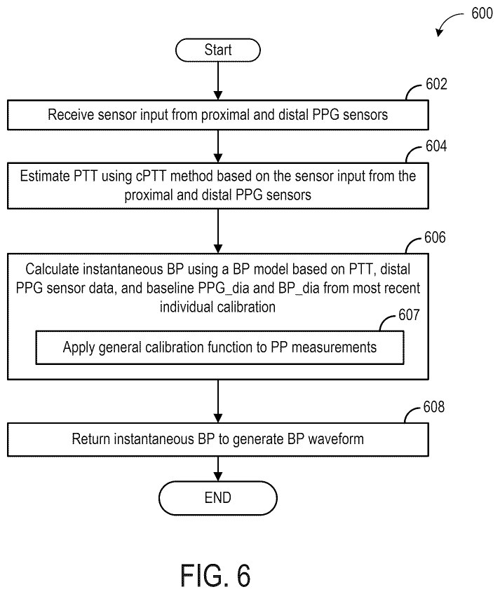

At 306, method 300 includes calculating an instantaneous BP of the patient based on outputs of the proximal PPG sensor and the distal PPG sensor, and the baseline PPG_{dia} amplitudes calculated at 304. The instantaneous BP may be measured at regular intervals t in real time. Calculating the instantaneous BP is described below in reference to FIG. 6.

At 308, method 300 includes generating a continuous BP waveform from the instantaneous BP measurements calculated at the regular intervals and displaying the continuous waveform on a display device (e.g., display unit 114) in real time.

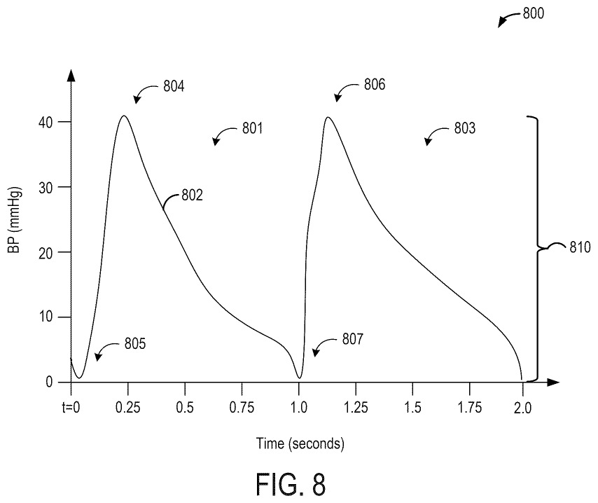

Referring briefly to FIG. 8, a PP waveform graph 800 is shown including a PP plot 802. (PP plot 802 may be easily converted to a similar BP plot for display on a display device). Pulse pressure is shown in millimeters of mercury (mmHg) on the y axis, and time is shown in seconds is shown on the x axis. PP plot 802 shows two cardiac cycles of a continuous PP waveform, which may be generated in accordance with the methods described herein based on an output of a proximal and a distal PPG sensor, as described in reference to FIGS. 2 and 3. Each cardiac cycle starts at a diastole of the cardiac cycle, climbs to a systole (e.g., peak PP) of the cardiac cycle, and descends to a diastole of a subsequent cardiac cycle. In FIG. 8, PP plot 802 includes a first cardiac cycle 801 and a second cardiac cycle 803. First cardiac cycle 801 includes a first diastole 805 (e.g., a starting point of first cardiac cycle 801) and a first systole 804. Second cardiac cycle 803 includes a second diastole 807 (e.g., a starting point of second cardiac cycle 803) and a second systole 806. An amplitude 810 of the continuous waveform indicates the PP of the patient at a respective systole, where the PP is equal to the PP at the systole minus the PP at the diastole. The continuous waveform indicates a PP of the patient at a given time t.

Returning to method 300, at 310, method 300 includes receiving and monitoring PPG_{dia} measurements made by the proximal PPG sensor and the distal PPG sensor. The PPG_{dia} measurements may be made during the diastole of each cardiac cycle, or at periodic intervals.

At 312, method 300 includes determining, for both the proximal PPG sensor and the distal PPG sensor, whether a difference between a PPG_{dia} measurement made at a respective interval and the reference baseline PPG_{dia} calculated during the step 304 is greater than a threshold value or percentage. For example, the threshold may be 1-2% change with respect to the baseline PPG_{dia}. In one example, the threshold could be introduced with a moving average filter to counter motion artifacts. The threshold could also be adaptive, to ensure that it prevents false positives during periods of high variability, while maintaining sensitivity during periods of low variability. If the difference is greater than the threshold value or percentage at either the proximal PPG sensor or the distal PPG sensor, it may be inferred that the PPG_{dia} may not be accurate. For example, the cuff may have slipped. When the accuracy of the PPG_{dia} is questioned, the individual calibration parameters of the proximal and distal PPG sensors may be recalculated for recalibration during the next instantaneous BP calculation.

A graphical representation of changes in blood pressure over time that shows a full range of pressure fluctuations of a cardiac cycle is referred to herein as an absolute blood pressure waveform. To ensure that a baseline deviation of the absolute blood pressure waveform does not exceed a regulatory acceptance range, such as ±5 mmHg, the output of the PP waveform during diastolic may be maintained within the regulatory acceptance range. That is, when the output of the PP waveform during the diastolic phase differs from a previous output by more than the regulatory acceptance range, the individual calibration parameters of the proximal and distal PPG sensors may be recalculated for recalibration during the next instantaneous BP calculation.

If at 312 it is determined that the difference is greater than the threshold value and/or the output of the PP waveform during the diastolic phase differs from the previous output by more than the regulatory acceptance range, then method 300 proceeds back to 304, and a recalculation of the individual calibration parameters is performed. Alternatively, if at 312 it is determined that the difference is not greater than the threshold value, then it is assumed that the PPG_{dia} measurements are still accurate, and method 300 proceeds to 314.

At 314, method 300 includes continuing to generate the continuous BP waveform from the proximal and distal PPG sensors, and continuing to display the continuous BP waveform on the display device until a next calibration is performed. In some examples, recalibrations may be periodically performed, in addition to the recalibrations performed at 312. By performing regular recalibrations, an accuracy of the PPG sensor measurements may be ensured.

Referring now to FIG. 4, a flowchart is shown illustrating an exemplary method 400 for generating a calibration function for calibrating an output of two PPG sensors, such as a proximal PPG sensor (e.g., proximal PPG sensor 208) and a distal PPG sensor (e.g., distal PPG sensor 210) of a PPG system (e.g., PPG system 10). The calibration function may be used to adjust parameters during calculation of an instantaneous BP as described in reference to FIG. 6. Method 400 may be performed on a population (e.g., a plurality) of patients.

Method 400 starts at 402, where method 400 includes collecting paired pulse pressure (PP) data from a plurality of cardiac cycles of a plurality of patients, using a traditional sensor using for monitoring blood pressure, and the two PPG sensors. For each patient of the plurality of patients, the LED powers of the proximal and distal PPG sensors, as well as the sensor contact pressure using the sphygmomanometer, should first be adjusted to get a highest PPG signal strength, based on a skin type of the patient. The LED powers, as well as the sensor contact pressure, may not be adjusted afterwards during the general calibration procedure.

In various examples, the traditional sensor may be an arterial sensor inserted into an artery of the patient, such as a brachial artery. In some examples, a different artery may be used, such as a carotid artery. However, inserting the traditional sensor into the brachial artery may result in a more direct comparison of a PP outputted by the traditional sensor with a PP estimated from the two PPG sensors. The arterial sensor may output instantaneous PPs continuously in real time. The PPs outputted by the arterial sensor may be used as a reference or target for PPs generated based on an output of the two PPG sensors. An accuracy of the PPs generated from the arterial sensor may be higher than an accuracy of the PP measured using a sphygmomanometer. However, in some embodiments, the traditional sensor may be a sphygmomanometer sensor, where reference PPs generated using the sphygmomanometer may be substituted for the reference PPs generated from the arterial sensor.

At 404, collecting the paired PP data includes receiving, from each patient of the plurality of patients, a first instantaneous PP from the arterial sensor. In some examples, a single first instantaneous PP may be acquired during each cardiac cycle of the patient. For example, the single first instantaneous PP may be acquired at the systole of the cardiac cycle (e.g., systoles 804 and 806 of FIG. 8), where the single first instantaneous PP may be a maximum PP for the cardiac cycle. In other examples, a plurality of first instantaneous PPs may be acquired in real time during consecutive time intervals of each cardiac cycle of the patient. For example, a first instantaneous PP may be acquired every 0.25 seconds. In such cases, a number of first instantaneous PPs acquired during the cardiac cycle may depend on technical limitations of the arterial sensor.

At 406, collecting the paired PP data includes calculating, at a same time as each first instantaneous PP acquired via the arterial sensor, a second instantaneous PP from an output of the proximal and distal PPG sensors. In other words, if a single first instantaneous PP is acquired at a systole of a cardiac cycle of the patient, then a single second instantaneous PP may be acquired at the systole of the relevant cardiac cycle (e.g., reflecting an amplitude of the waveform of the cardiac cycle, such as amplitude 810 of FIG. 8). If a first instantaneous PP is acquired at a first-time interval of a cardiac cycle of the patient, then a second instantaneous PP may be acquired at the same time interval of the relevant cardiac cycle. The second instantaneous PP may be calculated from the proximal and distal PPG sensors as described below in reference to FIG. 6.

At 408, method 400 includes pairing the first instantaneous PP and the second instantaneous PP, and storing the (first instantaneous PP, second instantaneous PP) pair in a vector in a memory of the PPG system (e.g., memory 109). Thus, the vector includes a plurality of pairs of PPs, where a first PP of the pair is generated using the arterial sensor, and the second PP of the pair is generated based on PPG sensor data. The vector may include PP pairs from a plurality of cardiac cycles of the plurality of patients. That is, multiple PP pairs may be generated from a single patient.

At 410, after the vector has been generated, method 400 includes performing a polynomial regression of the PP pairs included in the vector to define a calibration function. In examples where a single PP pair is acquired at a systole of the cardiac cycles, a single-variable polynomial regression may be performed, based on one data point for each cardiac cycle. Alternatively, in examples where PP pairs are acquired at continuous time intervals of the cardiac cycles, a multivariable polynomial regression may be performed. A multivariable polynomial regression model may combine multiple polynomial fits of the PP pairs, where each fit represents a different time interval of the cardiac cycle. The multivariable polynomial regression may provide a more comprehensive representation of blood flow dynamics, which could lead to a more accurate correlation with the arterial pressure waveform generated based on the output of the one or more PPG sensors. However, a disadvantage of the multivariable polynomial regression is that it may be more computationally intensive. Alternatively, a machine learning model could be used for multivariable polynomial regression, such as small feedforward networks, Recurrent Neural Networks (RNNs) or Long Short-Term Memory (LSTM) networks, although they might be even less energy efficient.

At 412, method 400 includes outputting the calibration function generated by the polynomial regression, and method 400 ends.

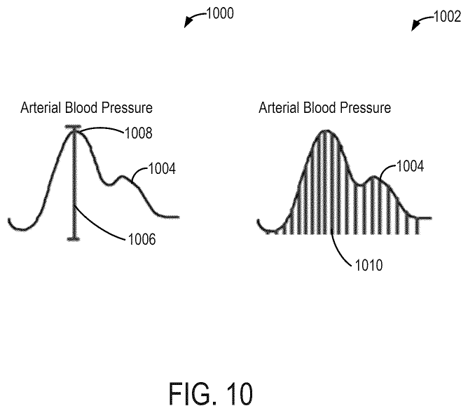

Turning to FIG. 10, the different approaches to the polynomial regression are described in reference to a first graphical depiction 1000 of a single-variable polynomial regression, and a second graphical depiction 1002 of a multivariable polynomial regression. In first graphical depiction 1000 where the single-variable polynomial regression is performed, the points of the plot to which the polynomial regression is applied are each defined by a maximum amplitude measurement 1006 taken of a cardiac cycle of an arterial blood pressure waveform 1004, where maximum amplitude measurement 1006 is measured at a systolic peak 1008 of the cardiac cycle, and a corresponding PP measurement from the arterial sensor, also taken at the systolic peak. Maximum amplitude measurement 1006 may be calculated by subtracting the diastolic baseline trend, aka DC component, from the amplitude value at systolic peak 1008, which sets the minimum value of the cycle to zero. This adjustment allows for correlation of pulse pressure waveforms without considering absolute signal values. Thus, when the single-variable polynomial regression is performed, for each point, maximum amplitude measurement 1006 is used to represent waveform data of the entire cardiac cycle. The maximum amplitude measurements would be aggregated across the plurality of subjects. In other words, the maximum amplitude data would be collected from all patients and combined into a single dataset. The single-variable polynomial regression can then be performed on the aggregated data, which helps in creating a more robust and generalizable model that reflects the overall trend across all subjects.

In contrast, in second graphical depiction 1002 where the multivariable polynomial regression is performed, rather than taking a single measurement at the systolic peak of arterial blood pressure waveform 1004, a plurality of amplitude measurements 1010 are taken at regular time intervals during the cardiac cycle, after subtracting the diastolic baseline trend, a.k.a., the DC component, from the whole cardiac signal. The maximum amplitude measurements would be aggregated across the plurality of subjects. The multivariable polynomial regression is then performed based on the aggregated plurality of amplitude measurements. Thus, when the multivariable polynomial regression is performed, a greater amount of the waveform data is used to fit a polynomial curve to the collected patient data. By using the greater amount of waveform data, an accuracy of the fit of the polynomial curve may be increased. As a result, an accuracy of a calibration of a PP calculation based on PPG sensors may be increased.

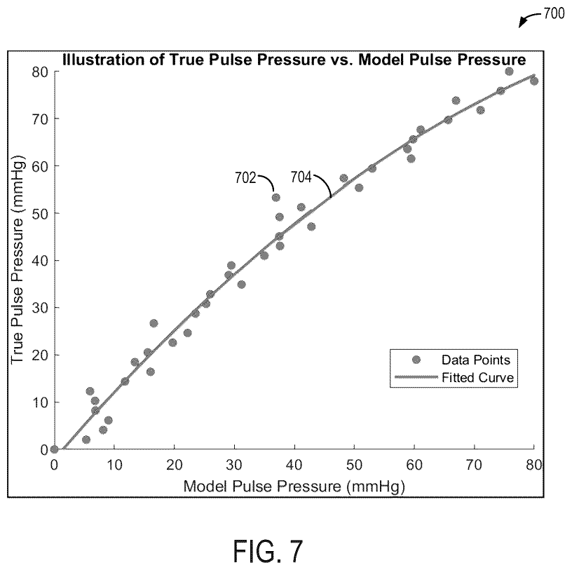

Now referring to FIG. 7, an exemplary graph 700 shows a plot of a plurality of points 702 representing PP pairs acquired as described in method 400. A first PP of each pair is shown on the x axis of graph 700, where the first PP is generated based on the output of the two PPG sensors of method 400 (in accordance with method 600 of FIG. 6). A second PP of each pair is shown on the y axis of graph 700, where the second PP is generated based on the output of the arterial sensor of method 400. A curve 704 has been fitted to the plurality of points 702, as a result of performing the polynomial regression. Curve 704 defines the general calibration function that may be applied to an output of the two PPG sensors to calibrate the output to a true PP based on the arterial sensor, described in reference to FIG. 6. Graph 700 shows points from a plurality of cardiac cycles of a plurality of subjects. For example, a first point may be based on data from a first cardiac cycle of a first patient; a second point may be based on data from a second cardiac cycle of the first patient; a third point may be based on data from a first cardiac cycle of a second patient; a fourth point may be based on data from a second cardiac cycle of the second patient; and so on.

Turning now to FIG. 5, a flowchart is shown illustrating an exemplary method for calculating calibration parameters for calibrating a PPG signal generated by a PPG sensor, such as a proximal PPG sensor (e.g., proximal PPG sensor 208) or a distal PPG sensor (e.g., distal PPG sensor 210) of a multi-parameter PPG system such as PPG system 10. Method 500 may be performed as part of method 300 of FIG. 3. The calculation and subsequent calibration using the calculated calibration parameters may be performed periodically during monitoring of a BP of a patient, as described above in reference to FIG. 3. During the monitoring, a cuff of a sphygmomanometer (e.g., cuff 204) may be fastened around an arm of the patient, as shown in FIG. 2. The cuff may be positioned such that the proximal PPG sensor and the distal PPG sensor are located on a brachial artery of the patient (e.g., brachial artery 206) and can sense a PP of the brachial artery.

Prior to starting method 500, the infrared LED power of an emitter of the multi-parameter PPG system should be adjusted for both the proximal PPG sensor and the distal PPG sensor, as well as the sensor contact pressure, to get a highest possible PPG signal strength for a type of skin of the patient. The LED powers of the proximal PPG sensor and the distal PPG sensor may be kept constant, as well as the sensor contact pressure, until recalibration occurs, at which point the LED power and the sensor contact pressure may be readjusted.

Method 500 starts at 502, where method 500 includes receiving a diastolic BP measurement from the sphygmomanometer. That is, a BP measurement may be automatically acquired from the sphygmomanometer at the diastole of a cardiac cycle of the patient.

At 504, method 500 includes receiving a plurality of diastolic PPG measurements from the distal and the proximal PPG sensors. The diastolic PPG measurements may be acquired from the distal and the proximal PPG sensors at various diastoles of the cardiac cycle (e.g., diastoles 805 and 807 of FIG. 8). Each diastolic PPG measurement (PPG_{dia}) represents a voltage of the light detected by a respective PPG sensor, reflected from the skin, originating from an infrared LED of the respective PPG sensor. This PPG_{dia} measurement is a non-pulsatile signal (e.g., DC component) right before the first pulsatile signals (e.g., AC component) become visible in the PPG signal. In one example, the cuff may be deflated slowly in increments, such that the PPG_{dia} value is an average of a short time window, instead of a single time point. That is, a diastolic PPG measurement may be acquired by each of the distal and the proximal PPG sensors at a precise moment when the cuff is deflated after the diastolic BP measurement is made, such that the diastolic PPG measurement is taken at a same time as the diastolic BP measurement.

At 506, method 500 includes calculating a baseline diastolic PPG measurement, where the baseline diastolic PPG measurement may be an average (e.g., mean) of the plurality of the diastolic PPG measurements received at 504. That is, a first plurality of diastolic PPG measurements acquired from the distal PPG sensor may be averaged to generate a first baseline PPG measurement for the distal PPG sensor, and a second plurality of diastolic PPG measurements acquired from the proximal PPG sensor may be averaged to generate a second baseline PPG measurement for the proximal PPG sensor. Both of the first baseline PPG measurement and the second baseline PPG measurement may be used in a subsequent calculation of the instantaneous BP described in reference to FIG. 6.

At 508, method 500 includes storing the diastolic BP measurement and the baseline diastolic PPG measurements for both of the distal and proximal PPG sensors in a memory of the PPG system (e.g., memory 109), for use in the subsequent calculation of the instantaneous BP as described below. The PPG signals from both proximal and distal sensors may be used to extract the Pulse Transit Time (PTT) and the PPG waveforms during the subsequent calculation of the instantaneous BP. That is, the diastolic PPG values of both proximal and distal PPG measurements may be fed into the general calibration function to generate a Pulse Pressure (PP) waveform, which is then combined with the diastolic blood BP measurement acquired at step 502 to obtain an absolute blood pressure waveform. Method 500 ends.

Referring now to FIG. 6, a flowchart is shown illustrating an exemplary method 600 for calculating an instantaneous BP of a patient being monitored, from PPG signals generated by a proximal PPG sensor and a distal PPG sensor, such as proximal PPG sensor 208 and distal PPG sensor 210 of FIG. 2. Method 600 may be performed as part of method 300 of FIG. 3.

At 602, method 600 includes receiving sensor input from the proximal PPG sensor and the distal PPG sensor. The proximal PPG sensor and the distal PPG sensor may be positioned within a cuff of a sphygmomanometer, as described in reference to FIG. 2.

At 604, method 600 includes estimating a pulse transit time (PTT) between cardiac cycles of the patient. PTT is defined as the time it takes a pulse pressure waveform to propagate through a length of the arterial tree, typically from the aorta to a peripheral arterial site. The PTT is typically measured between systoles of the cardiac cycle. A first time point is the systolic peak detected at the proximal PPG, and a second time point is the same systolic peak detected at the distal PPG at a later time. PTT can be used to determine a pulse wave velocity, which is related to vessel stiffness. The greater the stiffness, the higher the pulse wave velocity. Artery stiffness is modulated by factors such as age, atherosclerosis, and BP. At high BPs, artery walls are tense and hard, making the pulse wave travel faster (e.g., PTT is reduced). At low BPs, the artery walls have less tension, making the pulse wave travel slower (e.g., PTT is increased).

Rather than using systolic peak detection, the PTT may be estimated using the continuous pulse transit time (cPPT) method known in the art, which relies on cross-correlation inside of a moving window to estimate PTT continuously between two sites (e.g., the proximal PPG sensor and the distal PPG sensor). Using cPPT, a first time point is a systolic peak of the cardiac cycle detected by the proximal PPG sensor, and a second time point is the same systolic peak detected by the distal PPG sensor, where the PPT is the duration between the first time point and the second time point.

At 606, method 600 includes calculating an instantaneous PP based on the estimated PTT using a BP model, the sensor input from the proximal and distal PPG sensor, and proximal and distal baseline diastolic PPG measurement and diastolic BP measurement from a most recent calculation of individual calibration parameters (e.g., described in method 500). The instantaneous PP is generated using a BP model based on a BP equation derived from Laplace's law and the Moens-Korteweg equation for pulse wave velocity (PWV). The derivation of the BP model is described below.

Laplace's law relates an infinitesimal variation of a blood vessel radius δR to a variation of an internal blood pressure δBP, in accordance with equation (1):

$$δ BP = \frac{E \cdot h \cdot δ R}{{R}^{2}} \qquad ( 1 )$$

Where
   δBP is an infinitesimal variation of the internal blood pressure, in pascals (Pa) or newtons per square meter (N/m²);
   E is the modulus of elasticity of the blood vessel material, in pascals (Pa) or newtons per square meter (N/m²);
   h is the thickness of the blood vessel wall, in meters (m);
   δR is an infinitesimal variation of the blood vessel radius, in meters (m); and
   R is the radius of the blood vessel, in meters (m).

The Moens-Korteweg equation relates pulse wave velocity (PWV) in an elastic tube (e.g., a blood vessel) to its material properties and dimensions, in accordance with equation (2):

$$PWV = \frac{L}{PTT} = \sqrt{\frac{E \cdot h}{2 \cdot ρ \cdot r}} \qquad ( 2 )$$

Where
   PWV is the pulse wave velocity, in meters per second (m/s);
   L is the distance between measuring sites, in meters (m);
   PTT is the pulse wave transit time between measuring sites, in seconds(s);
   E is Young's modulus, in pascals (Pa) or newtons per square meter (N/m²);
   h is the thickness of the blood vessel wall, in meters (m);
   ρ is the blood density, in kilograms per cubic meter (kg/m³); and
   r is the internal radius of the blood vessel, in meters (m).

Replacing Moens-Korteweg in equation (2) into Laplace's law in equation (1) results in equation (3):

$$δ BP = 2 \cdot ρ \cdot {PWV}^{2} \cdot \frac{δ R}{R} \qquad ( 3 )$$

and integrating equation (3) over a cardiac cycle result in equation (4):

$$BP ( t ) - {BP}_{dia} = 2 \cdot ρ \cdot {PWV}^{2} \cdot log ( \frac{R ( t )}{{R}_{dia}} ) \qquad ( 4 )$$

Where
   BP(t) is the instantaneous blood pressure at time t, in pascals (Pa) or newtons per square meter (N/m²);
   BP_{dia} is the diastolic blood pressure, in pascals (Pa) or newtons per square meter (N/m²);
   BP(t)−BP_{dia} is the instantaneous pulse pressure PP;
   ρ is the blood density, in kilograms per cubic meter (kg/m³);
   PWV is the pulse wave velocity, in meters per second (m/s);
   R(t) is the instantaneous radius of the blood vessel at time t, in meters (m); and
   R_{dia} is the radius of the blood vessel during the diastole, in meters (m).

The PPG amplitude is a measure of the blood volume changes in the microvascular bed of tissue, which can be correlated to the changes in the arterial radius. By incorporating a calibration constant (C), a proportional relationship can be established between the PPG amplitudes and the arterial radii. The calibration constant (C) cancels out in the logarithm in equation (4), yielding the following equation (5):

$$BP ( t ) - {BP}_{dia} = 2 \cdot ρ \cdot {PWV}^{2} \cdot log ( \frac{PPG ( t )}{{PPG}_{dia}} ) \qquad ( 5 )$$

Where
   BP(t) is the instantaneous blood pressure at time t, in pascals (Pa) or newtons per square meter (N/m²);
   BP_{dia} is the diastolic blood pressure, in pascals (Pa) or newtons per square meter (N/m²);
   ρ is the blood density, in kilograms per cubic meter (kg/m³);
   PWV is the pulse wave velocity, in meters per second (m/s);
   PPG(t) is the instantaneous PPG amplitude at time t; and
   PPG_{dia} is the PPG amplitude during the diastole.

Equation (5) is essentially Pulse Pressure (PP) waveform, since PP(t)=BP(t)−BP_{dia}. For a continuous PWV estimation, the cPTT method may be utilized. To enhance the accuracy of the Photoplethysmogram Intensity Ratio (PIR), calculated as PIR(t)=PPG(t)/PPG_{dia}, it is recommended to average the PIR values obtained from both proximal and distal sensors. Equation (5) is divided by the conversion factor α to convert from pascals to millimeters of mercury (mmHg), resulting in equation 6:

$$PP ( t ) = \frac{2 \cdot ρ}{α} \cdot {( \frac{L}{PTT ( t )} )}^{2} \cdot log ( \frac{\frac{{PPG ( t )}_{proximal}}{{PPG}_{dia , proximal}} + \frac{{PPG ( t )}_{distal}}{{PPG}_{dia , distal}}}{2} ) \qquad ( 6 )$$

or in compact form

$$PP ( t ) = \frac{2 \cdot ρ}{α} \cdot {PWV ( t )}^{2} \cdot log ( \overline{PIR} ( t ) ) \qquad ( 7 )$$

Where
   PP(t) is the instantaneous pulse pressure at time t, in mmHg;
   ρ is the blood density, in kilograms per cubic meter (kg/m³);
   α is a pressure conversion factor (133.322 Pa/mmHg), converting pascals to millimeters of mercury;
   L is the distance between measuring sites, in meters (m);
   PTT (t) is the instantaneous pulse wave transit time between measuring sites at time t, in seconds(s);
   PPG(t)ₚᵣₒₓᵢₘₐₗ is the instantaneous PPG amplitude at the measuring site of the proximal PPG sensor at time t;
   PPG_{dia,proximal} is the PPG amplitude during the diastole at the measuring site of the proximal PPG sensor;
   PPG(t)_{distal} is the instantaneous PPG amplitude at the measuring site of the distal PPG sensor at time t;
   PPG_{dia,distal} is the PPG amplitude during the diastole at the measuring site of the distal PPG sensor; and
   P̅I̅R̅(t) is the average of instantaneous PPG intensity ratio between proximal and distal sensor at time t.

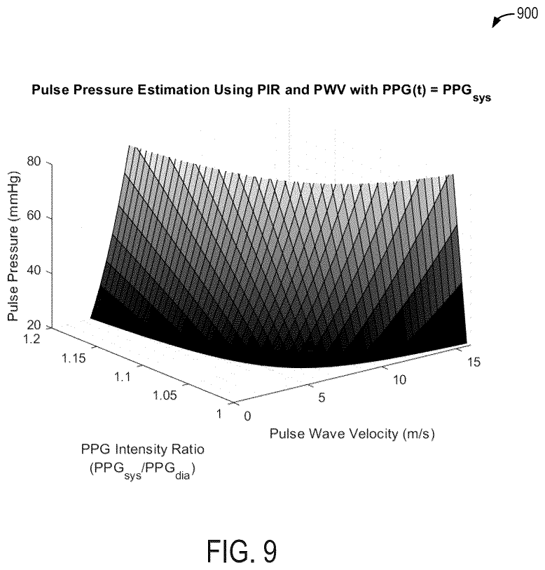

Thus, equation (6) combines the concepts of Laplace's law and the Moens-Korteweg equation to estimate PP based on PPG amplitude from the distal PPG sensor, allowing the PTT to dynamically reflect variations in the PPG signal and the PWV from both the proximal PPG sensor and the distal PPG sensor. BP_{dia} is updated in subsequent cardiac cycles. The derived model of PP estimation at a time t where PP=BP_{sys}−BP_{dia} is plotted in FIG. 9.

Referring briefly to FIG. 9, a PP estimation model 900 is shown at a time t where PP=BP_{sys}−BP_{dia}. PP estimation model 900 shows a relationship between a PPG Intensity Ratio (PIR) (e.g., systolic PPG amplitude divided by diastolic PPG amplitude), the PWV, and the PP at the time t. The Moens-Korteweg equation describes the relationship between PWV and blood pressure. However, it does not account for varying hemodynamics, leading to potential inaccuracies. By incorporating the PIR, which can be correlated to changes in the arterial radii, the dynamic behavior of blood vessels can be more accurately distinguished, potentially eliminating the reliance on recalibration in PP estimation. The PP model shown in FIG. 9 fuses PWV with PIR. PP represents the variable component of the absolute blood pressure waveform, with diastolic BP serving as the baseline offset. Consequently, a similar visualization can also be applied to data points within the absolute BP waveform.

Returning to method 600, at 607, calculating the instantaneous BP further comprises inputting an output of equation (7) into the general calibration function (calculated using method 400) to get a calibrated pulse pressure PP_{cal}:

$${PP}_{cal} ( t ) = C ( PP ( t ) )$$

Where C( ) is the general calibration function.

The baseline diastolic PPGs (PPG_{dia, proximal} and PPG_{dia, distal}) obtained from method 500 are then integrated into equation (7), and then and the diastolic BP (BP_{dia}) is added to equation (7) to obtain the instantaneous BPs, as shown in equation (8):

$$BP ( t ) = {BP}_{dia} + {PP}_{cal} ( t ) \qquad ( 8 )$$

Where PP_{cal}(t) is the instantaneous calibrated pulse pressure at time t, in mmHg.

At 608, the instantaneous BP calculated using equation (8) may then be returned, to generate a BP waveform for displaying on a display device, in accordance with method 300. Alternatively, in some embodiments, the PP waveform may be displayed on the display device, instead of the BP waveform. An advantage of displaying the PP waveform is that PP can be measured, in theory, without relying on the individual calibration described by method 500; thus, cuff inflation may not be performed. For the PP waveform, the baseline trend of the waveform (e.g., DC component) may be used for obtaining an instantaneous PPG_{dia}(t) value, since in this mode, the constant baseline PPG_{dia} is not obtained from the cuff calibration.

In some circumstances, such as during a general calibration, a shortened equation (9) of equation (7) for instantaneous Pulse Pressure may be used:

$$PP ( t ) = {( \frac{L}{PTT ( t )} )}^{2} \cdot log ( \overline{PIR} ( t ) ) \qquad ( 9 )$$

In the general calibration, PPG_{dia} has to be instantaneous and follow the baseline trend (e.g., DC component), since in the PP waveform, in its pure form, there is no individual calibration from which the static PPG_{dia} and the BP_{dia} are obtained. The equation (9) works for the general calibration, since all the other variables in the equation, (e.g., 2, ρ, α) are constants that can be fused with the constants of the polynomial. For example, in quadratic polynomial A+B*X(t)+C*X(t)², where X(t)=PP(t), the constants in the PP(t) can just be fused with the polynomial constants B and C. However, it may be wise not to fuse the L constant, the length between proximal and distal PPG sensors, to the polynomial; a new general calibration curve may have to be obtained if the length changes in the product, for example with different sized cuffs.

Thus, a method for monitoring blood pressure of a patient is proposed that relies on signals transmitted from two PPG sensors, where a cuff is periodically inflated for calibrating the signals rather than repeatedly inflated to make BP measurements. The method relies on a theoretical model that combines the concepts of Laplace's law and the Moens-Korteweg equation to estimate PP based on PPG Intensity Ratio (PIR) from a proximal and distal PPG sensor, allowing a PTT to dynamically reflect signal variations, and a PWV derived from both proximal and distal PPG sensors. The process of estimating an absolute blood pressure waveform includes concurrently measuring a baseline PPG signal (PPG_{dia}) via the proximal and the distal PPG sensor and a diastolic blood pressure (BP_{dia}) with a sphygmomanometer. If the baseline signal drifts beyond a certain threshold in either PPG sensor, automatic recalibration is initiated to maintain accuracy. To implement this model, the approach involves fitting a linear or polynomial curve to a plot of the theoretical model against clinical data. This fitted curve is then used for calibrating the measurements.

By monitoring the blood pressure using the PPG sensors, a discomfort of the patient may be reduced. A technical effect of monitoring the blood pressure using the PPG sensors is that an amount of computation and energy consumed by a blood pressure monitoring system may be reduced. That is, a first amount of energy consumed by the PPG sensors may be significantly less than a second amount of energy consumed by repeatedly inflating a sphygmomanometer cuff to take conventional blood pressure measurements. Further, an amount of computational and memory resources of the blood pressure monitoring system that are consumed by the proposed method may be reduced, with respect to alternative non-cuff-based blood pressure measurement methods. In contrast with other methods that may rely on PIR-arterial diameter and PIR-PP correlation models in conjunction with the Moens-Korteweg equation to enumerate arterial wall thickness's upper and lower bounds, or machine learning models, the proposed method relies on a simpler and more robust computational framework, which may result in reduced resource consumption and an increased battery life. The proposed method may also be less expensive and more resilient to motion artifacts than other methods that rely on piezoelectric ceramics or ultrasound sensors, which may additionally be less compact, heavier, and less energy efficient. A further technical effect is that by periodically calibrating the PPG signals using the cuff, an accuracy of the proposed method may be increased relative to other alternative methods.

The disclosure also provides support for a system for monitoring a blood pressure (BP) of a patient, comprising: a BP measurement device including a cuff, at least two photoplethysmography (PPG) sensors arranged in the cuff such that when the cuff is fastened around an arm of the patient, the at least two PPG sensors are positioned to sense a flow of blood in a brachial artery of the patient, and a controller and a memory storing instructions that when executed, cause the controller to: receive PPG signals from the at least two PPG sensors, calculate an instantaneous blood pressure (BP) of the patient based on the received PPG signals, display an arterial BP waveform generated from a plurality of instantaneous BP measurements on a display device, and periodically recalibrate the arterial BP waveform based on a reference signal measured by the at least two PPG sensors. In a first example of the system, the BP measurement device is a sphygmomanometer. In a second example of the system, optionally including the first example, the at least two PPG sensors include a proximal PPG sensor and a distal PPG sensor separated by a distance, the proximal PPG sensor closer to a heart of the patient than the distal PPG sensor. In a third example of the system, optionally including one or both of the first and second examples, the instantaneous BP is derived from a plurality of physiological parameters calculated from the received PPG signals. In a fourth example of the system, optionally including one or more or each of the first through third examples, the plurality of physiological parameters comprises one or more of a Pulse Wave Velocity (PWV), a Pulse Transit Time (PTT), and a PPG Intensity Ratio (PIR). In a fifth example of the system, optionally including one or more or each of the first through fourth examples, the PTT is calculated using a continuous pulse transit time (cPTT) method that relies on a cross-correlation inside of a moving window to estimate PTT continuously between the proximal PPG sensor and the distal PPG sensor. In a sixth example of the system, optionally including one or more or each of the first through fifth examples, the arterial BP waveform is recalibrated in response to a diastolic PPG measurement acquired from one or both of the proximal PPG sensor and the distal PPG sensor during a diastole of a cardiac cycle of the patient deviating from the reference signal by a threshold amount. In a seventh example of the system, optionally including one or more or each of the first through sixth examples, further instructions are stored in the memory that cause the controller to adjust a pulse pressure (PP) calculated based on the PPG signals using a general calibration function, the general calibration function determined by: for each subject of a plurality of subjects: acquiring PPG measurements at regular time intervals from the subject via the at least two PPG sensors, the PPG measurements acquired over various cardiac cycles, acquiring reference BP measurements from the subject at a same time as the PPG measurements, using a BP measurement device, generating a plurality of first estimated PPs of a plurality of cardiac cycles of the subject, the first estimated PPs calculated based on the acquired PPG measurements, generating a respective plurality of second true PPs calculated based on the reference BP measurements, and plotting the first estimated PPs against second true PPs for each cardiac cycle of the plurality of cardiac cycles, and after acquiring a plurality of plots from the plurality of subjects: combining the plurality of plots into a single overall plot, and performing a polynomial regression to fit a curve to the overall plot. In a eighth example of the system, optionally including one or more or each of the first through seventh examples, the polynomial regression is a single-variable polynomial regression where a measurement is made of a maximum amplitude of a cardiac cycle of an arterial blood pressure waveform of each subject of the plurality of subjects, the maximum amplitude measurements of each subject are aggregated, and the single-variable polynomial regression is performed based on the aggregated maximum amplitude measurement. In a ninth example of the system, optionally including one or more or each of the first through eighth examples, the polynomial regression is a multivariable polynomial regression where a plurality of amplitude measurements are made at regular time intervals during cardiac cycles of the arterial blood pressure waveform of each subject of the plurality of subjects, the plurality of amplitude measurements of each subject are aggregated, and the multivariable polynomial regression is performed based on the aggregated plurality of amplitude measurements. In a tenth example of the system, optionally including one or more or each of the first through ninth examples, the BP measurement device is an arterial sensor inserted into a brachial artery of the subject.

The disclosure also provides support for a method for continuous monitoring of a blood pressure (BP) of a patient, the method comprising: at regular time intervals during the continuous monitoring: receiving a first photoplethysmography (PPG) signal from a first PPG sensor positioned on an arm of the patient, receiving a second PPG signal from a second PPG sensor positioned on the arm, receiving diastolic PPG measurements acquired from both of the first PPG sensor and the second PPG sensor during a diastole of a cardiac cycle of the patient, calculating a Pulse Transit Time (PTT) of the patient based on the first PPG signal and the second PPG signal, calculating a Pulse Wave Velocity (PWV) of the patient based on the PTT, calculating an average PPG Intensity Ratio (PIR) of the cardiac cycle of a first PIR based on the first PPG signal and a second PIR based on the second PPG signal, calculating an instantaneous pulse pressure (PP) of the patient based on the PWV and the PIR, converting the instantaneous PP into an instantaneous BP measurement, displaying on a display device, in real time, an arterial BP waveform generated from a plurality of instantaneous BP measurements generated at the regular time intervals, and recalibrating the arterial BP waveform in response to either of the diastolic PPG measurements deviating from a reference diastolic PPG measurement measured by the first PPG sensor and/or the second PPG sensor by a threshold amount. In a first example of the method: the first PPG sensor and the second PPG sensor are integrated into a cuff of a cuff-based blood pressure device such that when the cuff is fastened around an arm of the patient, the first PPG sensor and the second PPG sensor are positioned to sense a flow of blood in a brachial artery of the arm, and the first PPG sensor and the second PPG sensor are separated by a distance along the arm such that the first PPG sensor is closer to a heart of the patient than the second PPG sensor. In a second example of the method, optionally including the first example, the method further comprises: calculating the PTT using a continuous pulse transit time (cPPT) method that relies on a cross-correlation inside of a moving window to estimate PTT continuously between the first PPG sensor and the second PPG sensor. In a third example of the method, optionally including one or both of the first and second examples, recalibrating the arterial BP waveform in response to either of the diastolic PPG measurements deviating from the reference diastolic PPG measurement further comprises: adjusting an LED power of the first PPG sensor and the second PPG sensor, as well as the sensor contact pressure, based on a skin type of the patient, measuring a diastolic BP of the patient using the cuff-based blood pressure device, measuring diastolic PPGs at both the first PPG sensor and the second PPG sensor, adjusting the instantaneous PP calculated from the first PPG signal and the second PPG signal based on the measured diastolic PPGs, and adding the measured diastolic BP to an updated PP waveform generated from the adjusted instantaneous PPs. In a fourth example of the method, optionally including one or more or each of the first through third examples, the method further comprises: after calculating the instantaneous PP of the patient based on the PWV and the PIR, adjusting the instantaneous PP by applying a general calibration function to the instantaneous PP, the general calibration function calculated by: prior to the continuous monitoring of the patient: for each subject of a plurality of subjects: acquiring PPG measurements at regular time intervals from the subject via the first PPG sensor and the second PPG sensor, the PPG measurements acquired over various cardiac cycles, acquiring reference BP measurements from the subject at a same time as the PPG measurements, using a BP measurement device, generating a plurality of first estimated PPs of a plurality of cardiac cycles of the subject, the first estimated PPs calculated based on the acquired PPG measurements, generating a respective plurality of second true PPs calculated based on the reference BP measurements, and plotting the first estimated PPs against second true PPs for each cardiac cycle of the plurality of cardiac cycles, and after acquiring a plurality of plots from the plurality of subjects: combining the plurality of plots into a single overall plot, and generating the general calibration function by performing a polynomial regression to fit a curve to the overall plot.

The disclosure also provides support for a method for generating an arterial blood pressure (BP) waveform of a patient, the method comprising: receiving instantaneous photoplethysmography (PPG) measurements generated at regular time intervals from the patient via a plurality of PPG sensors positioned on a brachial artery of the patient, generating a Pulse Pressure (PP) waveform from the instantaneous PPG measurements based on a Pulse Transit Time (PTT) of the patient measured between locations of the plurality of PPG sensors, and PPG Intensity Ratio (PIR) of the patient measured from the plurality of PPG sensors, and adjusting the instantaneous PP measurements by applying a general calibration function based on reference BP measurements collected in advance from a plurality of subjects. In a first example of the method, the method further comprises: generating the general calibration function by: for each subject of a plurality of subjects: acquiring PPG measurements at regular time intervals from the subject via the plurality of PPG sensors, the PPG measurements acquired over various cardiac cycles, acquiring reference BP measurements from the subject at a same time as the PPG measurements, using a BP measurement device, generating a plurality of first estimated PPs of a plurality of cardiac cycles of the subject, the first estimated PPs calculated based on the acquired PPG measurements, generating a respective plurality of second true PPs calculated based on the reference BP measurements, and plotting the first estimated PPs against second true PPs for each cardiac cycle of the plurality of cardiac cycles, and after acquiring a plurality of plots from the plurality of subjects: combining the plurality of plots into a single overall plot, and generating the general calibration function by performing a polynomial regression to fit a curve to the overall plot. In a second example of the method, optionally including the first example, the polynomial regression is a single-variable polynomial regression where a measurement is made of a maximum amplitude of a cardiac cycle of an arterial blood pressure waveform of each subject of the plurality of subjects, the maximum amplitude measurements of each subject are aggregated, and the single-variable polynomial regression is performed based on the aggregated maximum amplitude measurement. In a third example of the method, optionally including one or both of the first and second examples, the polynomial regression is a multivariable polynomial regression where a plurality of amplitude measurements is made at regular time intervals during cardiac cycles of the arterial blood pressure waveform of each patient of the plurality of patients, the plurality of amplitude measurements of each subject are aggregated, and the multivariable polynomial regression is performed based on the aggregated plurality of amplitude measurements.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc., are used merely as labels and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims and may include other examples that occur to those of ordinary skill in the art. Such other examples are intended to be within the scope of the claims if they have structural elements that do not differ from the literal language of the claims or if they include equivalent structural elements with insubstantial differences from the literal languages of the claims.

1. A system for monitoring a blood pressure (BP) of a patient, comprising:
a BP measurement device including a cuff;
at least two photoplethysmography (PPG) sensors arranged in the cuff such that when the cuff is fastened around an arm of the patient, the at least two PPG sensors are positioned to sense a flow of blood in a brachial artery of the patient; and
a controller and a memory storing instructions that when executed, cause the controller to:
receive PPG signals from the at least two PPG sensors;
calculate an instantaneous blood pressure (BP) of the patient based on the received PPG signals;
display an arterial BP waveform generated from a plurality of instantaneous BP measurements on a display device; and
periodically recalibrate the arterial BP waveform based on a reference signal measured by the at least two PPG sensors.

2. The system of claim 1, wherein the BP measurement device is a sphygmomanometer.

3. The system of claim 1, wherein the at least two PPG sensors include a proximal PPG sensor and a distal PPG sensor separated by a distance, the proximal PPG sensor closer to a heart of the patient than the distal PPG sensor.

4. The system of claim 3, wherein the instantaneous BP is derived from a plurality of physiological parameters calculated from the received PPG signals.

5. The system of claim 4, wherein the plurality of physiological parameters comprises one or more of a Pulse Wave Velocity (PWV), a Pulse Transit Time (PTT), and a PPG Intensity Ratio (PIR).

6. The system of claim 5, wherein the PTT is calculated using a continuous pulse transit time (cPTT) method that relies on a cross-correlation inside of a moving window to estimate PTT continuously between the proximal PPG sensor and the distal PPG sensor.

7. The system of claim 3, wherein the arterial BP waveform is recalibrated in response to a diastolic PPG measurement acquired from one or both of the proximal PPG sensor and the distal PPG sensor during a diastole of a cardiac cycle of the patient deviating from the reference signal by a threshold amount.

8. The system of claim 1, wherein further instructions are stored in the memory that cause the controller to adjust a pulse pressure (PP) calculated based on the PPG signals using a general calibration function, the general calibration function determined by:
for each subject of a plurality of subjects:
acquiring PPG measurements at regular time intervals from the subject via the at least two PPG sensors, the PPG measurements acquired over various cardiac cycles;
acquiring reference BP measurements from the subject at a same time as the PPG measurements, using a BP measurement device;
generating a plurality of first estimated PPs of a plurality of cardiac cycles of the subject, the first estimated PPs calculated based on the acquired PPG measurements;
generating a respective plurality of second true PPs calculated based on the reference BP measurements; and
plotting the first estimated PPs against second true PPs for each cardiac cycle of the plurality of cardiac cycles; and
after acquiring a plurality of plots from the plurality of subjects:
combining the plurality of plots into a single overall plot; and
performing a polynomial regression to fit a curve to the overall plot.

9. The system of claim 8, wherein the polynomial regression is a single-variable polynomial regression where a measurement is made of a maximum amplitude of a cardiac cycle of an arterial blood pressure waveform of each subject of the plurality of subjects, the maximum amplitude measurements of each subject are aggregated, and the single-variable polynomial regression is performed based on the aggregated maximum amplitude measurement.

10. The system of claim 9, wherein the polynomial regression is a multivariable polynomial regression where a plurality of amplitude measurements are made at regular time intervals during cardiac cycles of the arterial blood pressure waveform of each subject of the plurality of subjects, the plurality of amplitude measurements of each subject are aggregated, and the multivariable polynomial regression is performed based on the aggregated plurality of amplitude measurements.

11. The system of claim 8, wherein the BP measurement device is an arterial sensor inserted into a brachial artery of the subject.

12. A method for continuous monitoring of a blood pressure (BP) of a patient, the method comprising:
at regular time intervals during the continuous monitoring:
receiving a first photoplethysmography (PPG) signal from a first PPG sensor positioned on an arm of the patient;
receiving a second PPG signal from a second PPG sensor positioned on the arm;
receiving diastolic PPG measurements acquired from both of the first PPG sensor and the second PPG sensor during a diastole of a cardiac cycle of the patient;
calculating a Pulse Transit Time (PTT) of the patient based on the first PPG signal and the second PPG signal;
calculating a Pulse Wave Velocity (PWV) of the patient based on the PTT;
calculating an average PPG Intensity Ratio (PIR) of the cardiac cycle of a first PIR based on the first PPG signal and a second PIR based on the second PPG signal;
calculating an instantaneous pulse pressure (PP) of the patient based on the PWV and the PIR;
converting the instantaneous PP into an instantaneous BP measurement;
displaying on a display device, in real time, an arterial BP waveform generated from a plurality of instantaneous BP measurements generated at the regular time intervals; and
recalibrating the arterial BP waveform in response to either of the diastolic PPG measurements deviating from a reference diastolic PPG measurement measured by the first PPG sensor and/or the second PPG sensor by a threshold amount.

13. The method of claim 12, wherein:
the first PPG sensor and the second PPG sensor are integrated into a cuff of a cuff-based blood pressure device such that when the cuff is fastened around an arm of the patient, the first PPG sensor and the second PPG sensor are positioned to sense a flow of blood in a brachial artery of the arm; and
the first PPG sensor and the second PPG sensor are separated by a distance along the arm such that the first PPG sensor is closer to a heart of the patient than the second PPG sensor.

14. The method of claim 12, further comprising calculating the PTT using a continuous pulse transit time (cPPT) method that relies on a cross-correlation inside of a moving window to estimate PTT continuously between the first PPG sensor and the second PPG sensor.

15. The method of claim 13, wherein recalibrating the arterial BP waveform in response to either of the diastolic PPG measurements deviating from the reference diastolic PPG measurement further comprises:
adjusting an LED power of the first PPG sensor and the second PPG sensor, based on a skin type of the patient, and a contact pressure of the first PPG sensor and the second PPG sensor;
measuring a diastolic BP of the patient using the cuff-based blood pressure device;
measuring diastolic PPGs at both the first PPG sensor and the second PPG sensor;
adjusting the instantaneous PP calculated from the first PPG signal and the second PPG signal based on the measured diastolic PPGs; and
adding the measured diastolic BP to an updated PP waveform generated from the adjusted instantaneous PPs.

16. The method of claim 15, further comprising, after calculating the instantaneous PP of the patient based on the PWV and the PIR, adjusting the instantaneous PP by applying a general calibration function to the instantaneous PP, the general calibration function calculated by:
prior to the continuous monitoring of the patient:
for each subject of a plurality of subjects:
acquiring PPG measurements at regular time intervals from the subject via the first PPG sensor and the second PPG sensor, the PPG measurements acquired over various cardiac cycles;
acquiring reference BP measurements from the subject at a same time as the PPG measurements, using a BP measurement device;
generating a plurality of first estimated PPs of a plurality of cardiac cycles of the subject, the first estimated PPs calculated based on the acquired PPG measurements;
generating a respective plurality of second true PPs calculated based on the reference BP measurements; and
plotting the first estimated PPs against second true PPs for each cardiac cycle of the plurality of cardiac cycles; and
after acquiring a plurality of plots from the plurality of subjects:
combining the plurality of plots into a single overall plot; and
generating the general calibration function by performing a polynomial regression to fit a curve to the overall plot.

17. A method for generating an arterial blood pressure (BP) waveform of a patient, the method comprising:
receiving instantaneous photoplethysmography (PPG) measurements generated at regular time intervals from the patient via a plurality of PPG sensors positioned on a brachial artery of the patient;
generating a Pulse Pressure (PP) waveform from the instantaneous PPG measurements based on a Pulse Transit Time (PTT) of the patient measured between locations of the plurality of PPG sensors, and PPG Intensity Ratio (PIR) of the patient measured from the plurality of PPG sensors; and
adjusting the instantaneous PP measurements by applying a general calibration function based on reference BP measurements collected in advance from a plurality of subjects.

18. The method of claim 17, further comprising generating the general calibration function by:
for each subject of a plurality of subjects:
acquiring PPG measurements at regular time intervals from the subject via the plurality of PPG sensors, the PPG measurements acquired over various cardiac cycles;
acquiring reference BP measurements from the subject at a same time as the PPG measurements, using a BP measurement device;
generating a plurality of first estimated PPs of a plurality of cardiac cycles of the subject, the first estimated PPs calculated based on the acquired PPG measurements;
generating a respective plurality of second true PPs calculated based on the reference BP measurements; and
plotting the first estimated PPs against second true PPs for each cardiac cycle of the plurality of cardiac cycles; and
after acquiring a plurality of plots from the plurality of subjects:
combining the plurality of plots into a single overall plot; and
generating the general calibration function by performing a polynomial regression to fit a curve to the overall plot.

19. The method of claim 18, wherein the polynomial regression is a single-variable polynomial regression where a measurement is made of a maximum amplitude of a cardiac cycle of an arterial blood pressure waveform of each subject of the plurality of subjects, the maximum amplitude measurements of each subject are aggregated, and the single-variable polynomial regression is performed based on the aggregated maximum amplitude measurement.

20. The method of claim 18, wherein the polynomial regression is a multivariable polynomial regression where a plurality of amplitude measurements is made at regular time intervals during cardiac cycles of the arterial blood pressure waveform of each patient of the plurality of patients, the plurality of amplitude measurements of each subject are aggregated, and the multivariable polynomial regression is performed based on the aggregated plurality of amplitude measurements.
